(19) United States
(12) Patent Application Publication
(10) Pub. No.: US 2007/0259348 A1
(43) Pub. Date: Nov. 8, 2007

(54) Lyophilized pellets

(75) Inventors: Nikhil Phadke, Ann Arbor, MI (US); Michelle B. Manente, Ann Arbor, MI (US); Kalyan Handique, Ypsilanti, MI (US)

Correspondence Address: FISH & RICHARDSON P.C., PO BOX 1022, MINNEAPOLIS, MN, 55440-1022, US

(73) Assignee: HandyLab, Inc., Ann Arbor, MI (US)

(21) Appl. No.: 11/417,668

(22) Filed: May 3, 2006

Related U.S. Application Data
(60) Provisional application No. 60/737,519, filed on Nov. 16, 2005.

(30) Foreign Application Priority Data
May 3, 2005 (US) PCT/US2005/015345

(51) Int. Cl. C12Q 1/68; C12M 1/00; C12M 1/33; C12Q 1/00

(52) U.S. Cl. 435/6; 435/283.1; 435/306.1; 435/4

(57) ABSTRACT

Lyophilized pellets, suitable for use in a microfluidic device, and a method for preparing the same are described. The lyophilized pellets contain various biological reagents, or microparticles, and a cryoprotectant. The lyophilized pellets have a high degree of sphericity and are in the size range 0.5 to 35 μL. The pellets are prepared by dispensing drops of reagent solution on to a cryogenically cooled plate, followed by subjecting to a vacuum.

### CLAIM OF PRIORITY

This application claims the benefit of priority of U.S. provisional application Ser. No. 60/737,519, filed Nov. 16, 2005, and also claims priority to international application serial no. PCT/US2005/015345, filed May 3, 2005, both of which are incorporated herein by reference in their entirety.

### FIELD OF THE INVENTION

The present invention generally relates to lyophilized pellets. The present invention particularly relates to lyophilized pellets that are suitable for use in a microfluidic device, and methods for making the same.

### BACKGROUND

Lyophilization—often called freeze-drying—is an effective process for converting a biological reagent into a form that is convenient to handle, but which does not result in a concomitant loss of activity of the biological reagent. Lyophilization involves removing water content by sublimation from a frozen mixture, usually under vacuum, in such a manner that the concentration of non-aqueous ingredients is increased. Lyophilized materials typically have a porous structure that arises when bubbles of water vapor expand within the material during the process. Although lyophilized materials may be delicate to handle, they are usually easily reconstituted into solution forms of their ingredients and are much lighter than corresponding solution forms. Lyophilization practice and equipment are described in, e.g., Lyophilization—Introduction and Basic Principles, T. A. Jennings, (CRC Press LLC, Boca Raton, Fla.), incorporated herein by reference.

Lyophilization has been used to create pellets as small as the constituents of fine powders (sub-micron in diameter) and larger pellets whose diameters are 3-10 mm. However, hitherto it has been difficult to make lyophilized pellets in the 0.5 to 3 mm diameter (approx. 0.1 to 15 microliter) range. Such pellets would, if available, be useful for the practical delivery of reagents in microfluidic systems, where the volumes of reagents are on the scale of a few microliters, and where reaction chambers are only a few millimeters in dimension.

Nevertheless, existing techniques for creating lyophilized pellets are not easily adapted to create pellets in the 0.1 to 15 microliter range and in a form suitable for deployment in microfluidic devices.

### SUMMARY

The present invention relates to a lyophilized pellet and a method of making the pellet. The pellet preferably comprises one or more reagents. Even more preferably the reagents are biological reagents, such as enzymes. Still more preferably the reagents (e.g., primers, control plasmids, polymerase enzymes) are those deployed in the polymerase chain reaction (PCR), or in steps ancillary to performing PCR, such as sample preparation. Thus, the lyophilized pellets of the present invention are also suitable for lysing cells when the pellets include lysing reagents (e.g., enzymes). In particular, lyophilized pellets of the present invention that contain lytic enzymes (specific to a particular bacterium, for example, or non-specific) can lyse cells to release polynucleotides. The lyophilized pellets can include additionally, or in the alternative, enzymes (e.g., proteases) that degrade proteins, nucleases that degrade a particular nucleic acid (e.g., RNAses or DNAses), or lipases that degrade lipids. The lyophilized pellets of the present invention are especially suitable for deploying in a microfluidic device.

In some embodiments, the lyophilized pellets include multiple smaller particles, such as microspheres, each having a plurality of ligands attached to them. Such ligands associate preferentially with biomolecules such as polynucleotides, as compared to their propensity to associate with other species, for example, PCR inhibitors. Such lyophilized pellets are suitable for lysing cells when the lyophilized pellets include additionally lysing reagents (e.g., enzymes). Preferably such reagents lyse cells to release polynucleotides. The polynucleotides from the cells become associated with ligands bound to the smaller particles. The lyophilized pellets can also include enzymes (e.g., proteases) that degrade proteins.

Cells can be lysed by combining a solution of the cells with the lyophilized pellets to reconstitute the pellets. The reconstituted lysing reagents from the pellets lyse the cells. During lysis, the solution may be heated (e.g., radiatively using a lamp, such as a heat lamp).

The present invention further includes a method for making lyophilized pellets, comprising forming a solution of one or more reagents and a cryoprotectant (e.g., a sugar or poly-alcohol). The solution is deposited dropwise on a chilled hydrophobic surface, e.g., a diamond film, a silicon-oxide film, or polytetrafluoroethylene (PTFE) surface. Preferably the surface is a composite of diamond and silicon oxide. The pellets freeze and are subjected to reduced pressure (typically while still frozen) for a time sufficient to remove (e.g., sublimate) the solvent.

The present invention also includes a lyophilized pellet, comprising: a cryoprotectant; a biological reagent in a class selected from the group consisting of: enzymes, proteins, primers, fluorogenic probes, plasmids, polypeptides, nucleic acids, and the nucleotides dATP, dGTP, dCTP, dTTP and optionally dUTP; a buffering agent such as Tris; and salts such as KCl, MgCl₂, and (NH₄)₂SO₄; wherein the lyophilized pellet has a volume in the range 0.1 to 35 μL, and preferably in the range 0.5 to 25 μL, more preferably in the range 1 to 15 μL, and even more preferably in the range 2 to 10 μL.

The present invention also includes a lyophilized pellet, comprising: a cryoprotectant; and a plurality of microspheres having a concentration in the range of 10³ to 10¹³ microspheres per mL, and preferably in the range 10⁶ to 10¹⁰ microspheres per mL, wherein the lyophilized pellet has a volume in the range 0.5 to 35 μL, and preferably in the range 0.5 to 25 μL, more preferably in the range 1 to 15 μL, and even more preferably in the range 2 to 10 μL.

The present invention also includes a method for making a lyophilized pellet, comprising: introducing a liquid composition into a dispensing tip; positioning the dispensing tip above a cryogenically cooled plate, wherein the plate has a hydrophobic surface, and wherein the tip is in close proximity to the surface; dispensing a droplet of the liquid composition from the tip on to the surface; removing the tip away from close proximity to the surface so that the droplet remains in contact with the surface; maintaining the droplet in contact with the surface for such time as the droplet freezes to form a frozen droplet; and placing the frozen droplet into a lyophilizer for a time sufficient to produce a lyophilized pellet.

The present invention still further includes a lyophilized pellet made by the foregoing method and having a sphericity between 0.75 and 1. The invention also includes such a lyophilized pellet, additionally containing between about 1 and about 10¹⁰ microspheres in a pellet.

The present invention even further includes an apparatus for preparing lyophilized pellets, comprising: a cryogenically cooled plate having a hydrophobic surface; a dispensing tip configured to dispense a droplet of liquid onto the hydrophobic surface so that the droplet freezes; a dispensing system configured to position the dispensing tip above and in proximity to the hydrophobic surface; and a chamber enclosing at least the hydrophobic surface, configured to apply conditions of temperature and pressure sufficient to lyophilize the droplet.

The present invention additionally includes a microfluidic cartridge, comprising: a reagent inlet, wherein are situated one or more lyophilized pellets that each contain one or more reagents; a lysis chamber, wherein are situated one or more lyophilized pellets that each contain one or more lysis reagents; at least one valve; at least one gate; at least one outlet; at least one vent; and at least one channel configured to permit fluid to pass between the inlet, chamber, and outlet; wherein the one or more lyophilized pellets have a composition as further described herein.

The details of one or more embodiments of the invention are set forth in the accompanying drawings and the description herein. Other features, objects, and advantages of the invention will be apparent from the description and drawings, and from the claims.

### DESCRIPTION OF DRAWINGS

FIG. 1 illustrates a method of dispensing a droplet from a tip on to a hydrophobic surface, thereby forming an almost spherical pellet.

FIG. 2A and FIG. 2B illustrate a lyophilization tray (plan views), and in particular a container and lid for pellet lyophilization and storage.

FIGS. 3A and 3B show a side view of a lyophilization tray, during lyophilization (FIG. 3A) and a sealed container wherein a lid or seal is attached after lyophilization (FIG. 3B).

FIGS. 4 and 5 depict exemplary apparatus for producing lyophilized pellets.

FIG. 6 depicts a flow chart for a method of creating lyophilized pellets.

FIGS. 7A and 7B depict an exemplary microfluidic cartridge for using lyophilized pellets.

Like reference symbols in the various drawings indicate like elements.

### DETAILED DESCRIPTION

The lyophilized pellets of the present invention are especially suitable for delivering compositions that include microspheres. Other compositions have just one or more biological reagents such as enzymes. Still other lyophilized pellets contain a combination of microparticles and one or more biological reagents.

The methods of the present invention permit one to lyophilize combinations of biological materials and microparticle suspensions without significant loss of activity of any of the individual components.

The methods of the present invention permit one to lyophilize mixtures of biological materials (including enzymes, other proteins, primers, fluorogenic probes, plasmids, etc.) in the form of master mixes, in such a way that the biological materials retain their biochemical activity, and preferably their entire activity.

The methods of the present invention also permit one to lyophilize microparticle suspensions, e.g., suspensions of polystyrene latex microspheres or magnetic microspheres (both with or without activated surface chemistries). Such microparticles are examples of affinity materials.

The lyophilized pellets of the present invention may be used in a process for determining the presence of one or more polynucleotides in a sample. As such, the lyophilized pellets typically include several reagents. In some embodiments, the lyophilized pellets include one or more compounds used in a reaction for determining the presence of a polynucleotide and/or for increasing the concentration of the polynucleotide. For example, lypophilized pellets can include one or more enzymes for amplifying the polynucleotide as by PCR.

Microspheres, if used with the present invention, are preferably coated with one or more polycationic materials. The polycationic materials are preferably selected from the group consisting of: poly-D-lysine; polyethyleneimine (PEI); poly-DL-ornithine; and poly-histidine.

Preferably such a polycationic material is poly-D-lysine having an average molecular weight from 1,000-4,000 Daltons. Still more preferably, the polycationic material is poly-D-lysine having an average molecular weight of 1770 Daltons.

If the polycationic material is PEI, its molecular weight is preferably in the range 600-800 Daltons. It is found that branched PEI is more effective for forming microspheres to be lyophilized than linear PEI of an equivalent molecular weight.

If the polycationic material is poly-DL-ornithine, its molecular weight is preferably in the range 12,000-30,000 Daltons.

Poly-histidine is particularly preferred for RNA applications because, in its application to RNA, it utilizes lower binding, wash, and release pH's than is applicable to comparable DNA applications. Poly-histidine binds RNA at a pH of approximately 4, can be washed at a pH of 4-5 and released at a pH 8-9.

Typically, the lyophilized pellets of the present invention have an average volume of between about 0.1 microliters and about 5 microliters (e.g., about 4 microliters, about 3 microliters, about 2 microliters, or about 1 microliter). It is to be understood that the term ‘about’, as applied to a pellet having a volume v, means that the pellet has a volume of v±0.5 microliters. Preferably, production pellets have a volume of 2 μL, though pellets as small as 0.5 μL can be employed.

Typically, the lyophilized pellets, although preferably spherical in shape, may be non-uniform in diameter, and have an average diameter of between about 0.5 mm and about 5 mm (e.g., about 4 mm, about 3 mm, about 2.5 mm, about 2 mm, or about 1 mm). It is to be understood that the term ‘about’, as applied to a pellet having an average diameter d, means that the pellet has an average diameter of d±0.5 mm.

The lyophilized pellets made by the method of the present invention are advantageous because they are spherical to a high degree of uniformity. This enables the pellets to be more efficiently handled by vacuum pick-up methods. Sphericity, s, of a pellet can be measured by a parameter that is defined as a ratio of ratios. In particular, it is the ratio of the surface area to volume of the pellet divided by the ratio of the surface area to volume of an idealized sphere having the same displacement volume as the pellet. Thus, considering the ratio of surface area to volume of a perfect sphere of volume V, to be s₀, and the ratio of the surface area to volume of a pellet of the present invention also having volume V to be sₚ, the sphericity of the pellet is given by the formula: s=sₚ/s₀. Preferably the pellets of the present invention have a sphericity in the range 0.7-1.0 (where 1.0 indicates a perfectly spherical pellet). Even more preferably, the sphericity is in the range 0.9-1.0.

It is also preferable that a population of pellets produced by the methods of the present invention have average diameters within a close range, for example within ±0.5 mm of one another. Thus, for example, a population of pellets is composed of pellets, all of whose average diameters lie in the range 3±0.5 mm.

In an exemplary embodiment, a population of lyophilized pellets has an average volume of about 2 microliters, and an average diameter of about 1.35 mm. The average diameter of preferred 2 μL PCR pellets is about 1.3-1.7 mm.

Cryoprotectants generally help increase the stability of the lyophilized pellets and help prevent damage to reagents in the pellets (e.g., by preventing denaturation of enzymes during preparation and/or storage of the pellets, and also during reconstitution of the pellet). Furthermore, a cryoprotectant also gives physical stability to the pellets. Some cryoprotectants also prevent oxidation of the reagents. Preferably the cryoprotectant is the sugar, trehalose. In certain embodiments, the cryoprotectant is used in combination with a bulking agent such as dextran. Suitable cryoprotectants also include glycols such as ethylene glycol, propylene glycol, and glycerol. In some embodiments, the cryoprotectant comprises one or more sugars (e.g., one or more disaccharides, such as trehalose, melizitose, raffinose) mixed with one or more poly-alcohols (e.g., mannitol, sorbitol).

The pellets may also contain one or more bulking agents such as dextran. Bulking agents help to maintain rigidity of the lyophilized pellets. Being inert, they also keep the reagent molecules physically separate from each other, thus reducing their ability to react with other molecules.

Three preferred combinations of ingredients found in lyophilized pellets of the present invention are as follows: In a first embodiment, in pellets suitable for carrying out PCR, the pellets comprise: a cryoprotectant; and a PCR reagent mix; optionally salts such as KCl, MgCl₂, and (NH₄)₂SO₄; optionally a buffering agent; and optionally a bulking agent. In a variation of the first embodiment, the pellets consist of the foregoing reagents, and in still another variation of the first embodiment, the pellets consist essentially of the foregoing reagents. PCR reagent mixes suitable for use in the first embodiment are familiar to one of ordinary skill in the art and preferably include: at least one enzyme; at least one protein; at least one primer; at least one fluorogenic probe; at least one plasmid; at least one polypeptide; at least one optional nucleic acid that function as a non-specific control; and at least one nucleotide such as dATP, dGTP, dCTP, dTTP or dUTP.

In a second embodiment, the pellets are suitable for, e.g., DNA capture applications, and the pellets comprise: a plurality of microspheres having a concentration in the range of 10³ to 10¹³ per mL, a cryoprotectant, and optionally a bulking agent, wherein the microspheres have a binding agent, such as a ligand, bound to their exteriors. In a variation of the second embodiment, the pellets consist of the foregoing reagents, and in still another variation of the second embodiment, the pellets consist essentially of the foregoing reagents.

In a third embodiment, the pellets contain a mixture of enzymes, as might be used in sample preparation, for example in connection with microfluidic analysis. Such pellets comprise: a mixture of enzymes, and a cryoprotectant, optionally a salt, optionally a buffer, and optionally a bulking agent. In a variation of the third embodiment, the pellets consist of the foregoing reagents, and in still another variation of the third embodiment, the pellets consist essentially of the foregoing reagents. Preferably the mixture of enzymes is composed of more than two enzymes, e.g., 3, 4, 5, 7, 10, or more different enzymes. Preferably the mixture of enzymes includes at least one enzyme selected from the group consisting of: RNase A; pronase; proteinase K; and mutanolysin.

In yet another embodiment, the second and third embodiments are conflated, and pellets comprise: a cryoprotectant; and a mixture of enzymes used in sample preparation; and a plurality of microspheres for nucleic acid capture. Other biomolecules that could go into these pellets include but are not limited to: specific or non specific nucleic acids as external controls, e.g., DNA plasmids, intact genomic DNA of another organism chosen as a control, protected RNA's, PNA's, LNA's or other modified nucleic acids. Additional capture materials can also be included, anchored to a plurality of microspheres, such as antibodies, aptamers, lectins, or other oligonucleotides having specific or non-specific affinities for a biomolecule of interest.

In exemplary embodiments for use in systems for determining the presence of a biological agent—such as Group B Streptococcus (GBS)—in a sample, the lyophilized pellets include one or more of: a cryoprotectant, one or more salts, one or more primers (e.g., GBS forward and reverse primers (known as GBS Primer F and GBS Primer R)), one or more probes (e.g., GBS Probe—FAM, where FAM denotes a fluorescence color), one or more internal control plasmids, one or more specificity controls (e.g., Streptococcus pneumoniae DNA as a cross-reactivity control for PCR of GBS), one or more PCR reagents (e.g., dNTPs and/or dUTPs), one or more blocking or bulking agents (e.g., non-specific proteins such as bovine serum albumin (BSA), RNAseA, or gelatin), and a polymerase (e.g., glycerol-free Taq Polymerase). It is to be understood that, preferably, all such ingredients as are present are found in any given pellet. It would be understood by one of ordinary skill in the art that such a formulation, suitable for determining the presence of GBS in a sample, can be used for amplification of other polynucleotides upon use of other components (e.g., other primers and/or specificity controls). Examples include, but are not limited to, pathogens such as Yersinia pestis (plague), Erwinia herbicola (a plant pathogen often used as a plague stimulant), Bacillus anthracis, and Bacillus globigii (an anthrax stimulant), Listeria monocytogenes, E. coli O157, and Herpes Simplex Virus 1 & 2.

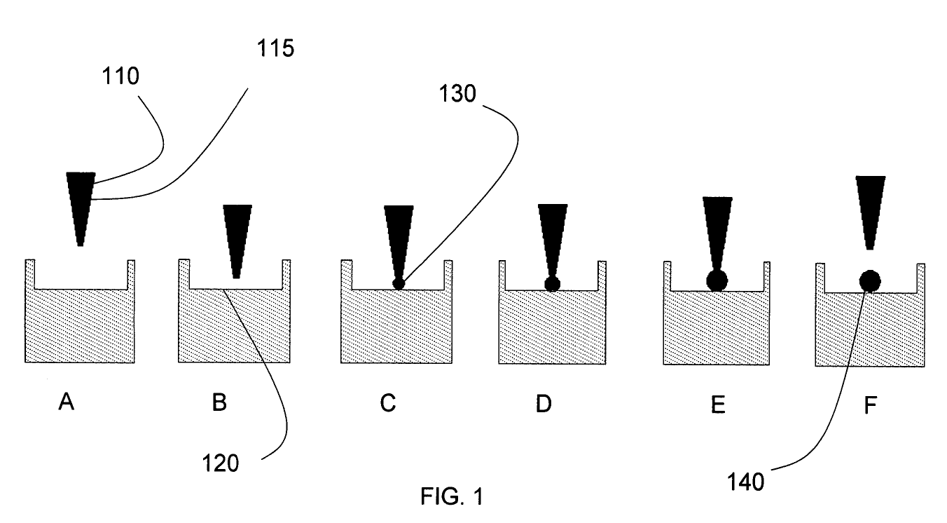

Lyophilized pellets according to the present invention are preferably prepared by the following method, as exemplified in FIG. 1 at views A-F. Typically, reagents to be placed in the lyophilized pellets are combined with a solvent (e.g., water) and a cryoprotectant to make a solution 115. The solution is then placed by a dispensing method, (e.g., in discrete aliquots such as drops, such as by a pipette 110), onto a chilled hydrophobic surface 120 (see FIG. 1 at panels A-C). Thus, for example, the solution is introduced into a dispensing tip, and the dispensing tip is positioned above the surface 120, prior to dispensing a droplet of liquid on to the surface. Preferably the liquid is agitated during at least the period of time it is being introduced into the dispensing tip, the time that the dispensing tip is being positioned, and the time that the liquid is being dispensed. The pipette 110 is preferably controlled by a robotic dispensing system that can control its vertical motion as well as its motion in a horizontal plane parallel to the hydrophobic surface so that it can dispense several droplets on various parts of the surface. The tip of pipette 110 is preferably kept far (e.g., ˜1-10 cm) from the hydrophobic surface until it is desired to dispense liquid. A robotically controlled dispensing system may be multiplexed, having a number of dispensing tips, say 4, 8, 10, 20, or 24, all able to dispense solution simultaneously, and arranged in an array or in a line.

The temperature of the hydrophobic surface is adjusted so that the liquid being dispensed does not freeze in the dispensing head 110 (such as a robotic head, or pipettor tip) but rapidly freezes from bottom up within seconds of contact with the surface (see FIG. 1 at D) so that there is no significant loss due to evaporation and no significant change in the physical shape of the dispensed reagent pellet. The solution freezes as discrete pellets 140. In this way, a pellet 140 that is almost spherical is created. Such control is achieved based on distance from (height above) a bath of cooling agent such as liquid nitrogen. The dispensing is performed in such a manner that the tip never touches the hydrophobic surface. The control is further such that the tip does not stick (by freezing) to the dispensed liquid reagent, but the droplet is transiently bound on either side by the pipette tip and the hydrophobic surface, as in FIG. 1 at C, D and E. In particular, the pipette tip is positioned about 0.5 to 1.5 mm, and preferably 0.5-1.0 mm, from the hydrophobic well surface during the start of liquid dispense (see FIG. 1 at B). Accordingly, the dispense velocity is slow and controlled. As the liquid drop 130 emerges from the pipette tip and touches the cold hydrophobic surface, the drop freezes from the bottom upwards (FIG. 1 at D). More liquid is continued to be dispensed before the tip is moved away from the surface, until the entire liquid volume (0.5-35 μl, and preferably 2-25 μl) is dispensed (FIG. 1 at E). For example, for a 2 microliter pellet, the dispense time is slightly less than the freezing time (1-2 sec). For a 25 microliter pellet, the dispense time is 2-3 sec compared to the freezing time of 2-5 sec.

Preferred examples of a hydrophobic surface 120 are a diamond film, or a polytetrafluorethylene (PTFE) surface, in particular a Teflon®-coated glass slide, or a mixture of diamond and SiO₂, the ratio of which may be adjusted to achieve different degrees of hydrophobicity. The hydrophobic surface is preferably made by a deposition method such as chemical vapor deposition (CVD) onto a metallic slide surface. Another method of making a surface is laser deposition of carbon/silicon dioxide coating material.

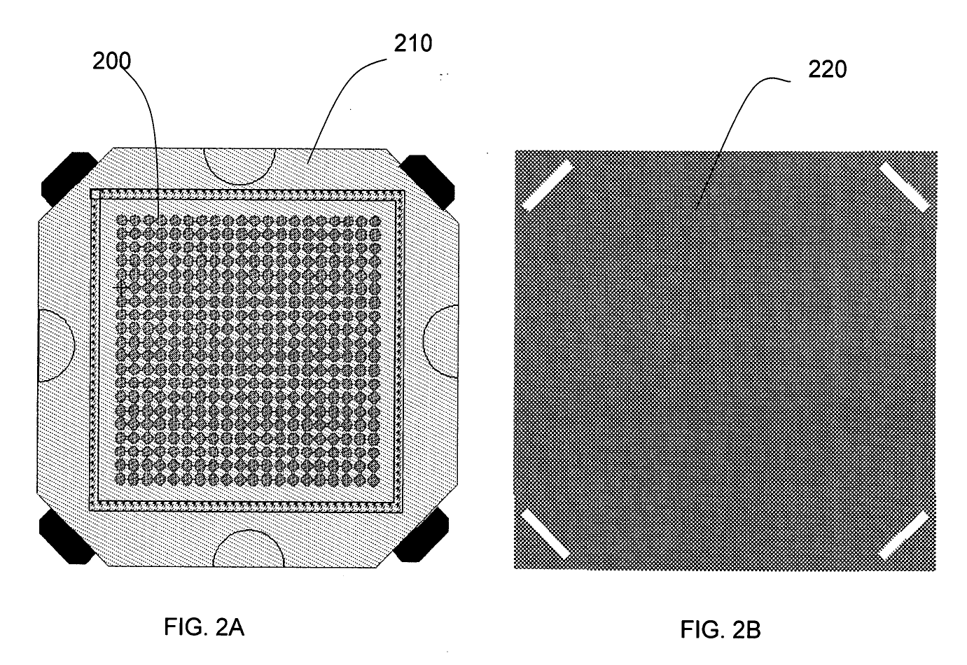

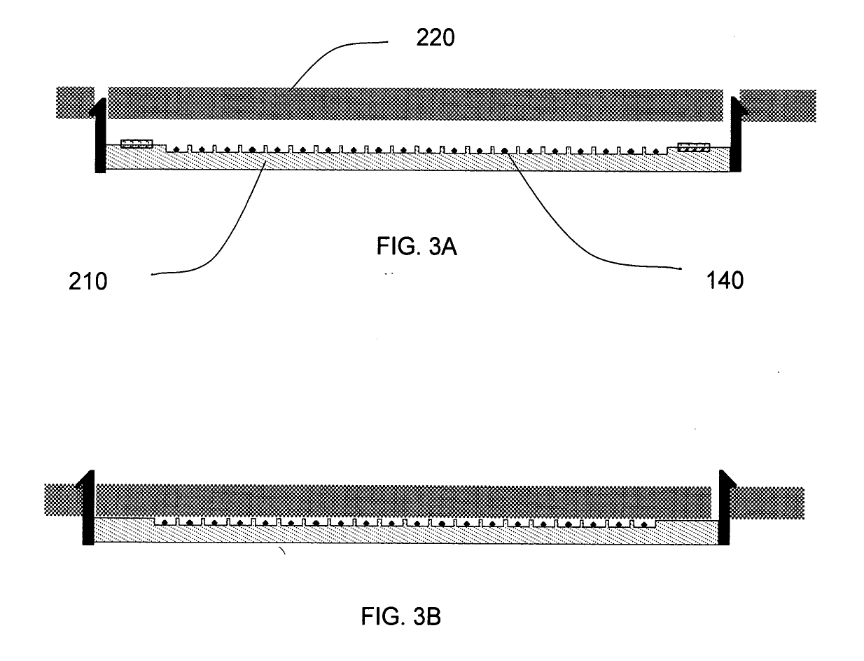

FIGS. 2A and 2B show a plan view of such a hydrophobic surface showing how a number of pellets can be accommodated, separately from one another, in an array of wells 200 (also referred to as ‘micro-chambers’), disposed upon a base 210. FIGS. 3A and 3B show a side-on view of the surface in FIGS. 2A and 2B. The hydrophobic surface is preferably essentially flat, by which it is meant that it is preferably smooth so that the pellets do not adhere to it, and is preferably oriented horizontally. The number of wells 200 is variable, and preferably is a number that facilitates use of a multi-drop dispenser. The number may be around 100, such as 96, or 125, or may be as high as 400, or even 1,000. The number will depend upon the size of pellets to be dispensed, as well as the available size of lyophilizer.

Both wells 200 and base 210 are made from the same material, having the hydrophobic surface 120. Preferably the wells are chilled by disposing the entire base 210 over a liquid bath (not shown) containing a cryogenic agent such as liquid nitrogen. In general, the temperature of the surface is reduced to near the temperature of the cryogenic agent. Thus, by being placed in proximity to a source of liquid nitrogen (whose temperature is typically about −196° C., the surface is preferably between about −65° C. to −180° C., more preferably between about −100° C. and about −150° C.). The method also works if the temperature is in the range −50° C. to −100° C. Optionally, the surface is cooled by immersing the hydrophobic surface in liquid nitrogen and equilibriating the surface with the liquid nitrogen, prior to the dispensing.

The frozen pellets 140 are introduced into a lyophilization apparatus and subjected to a vacuum while still frozen for a pressure and time sufficient to remove the solvent (e.g., by sublimation) from the pellets, thereby forming lyophilized pellets (FIG. 3A). A lid 220 (see FIGS. 2 and 3) is constructed so that it fits over the base 210 and can seal the lyophilized pellets from the environment (FIG. 3B). The period of residency in the lyophilizer sufficient to produce lyophilized pellets will vary according to pellet size and composition, but is typically about 20-40 hours, preferably about 24-30 hours, and even more preferably, about 25-27 hours.

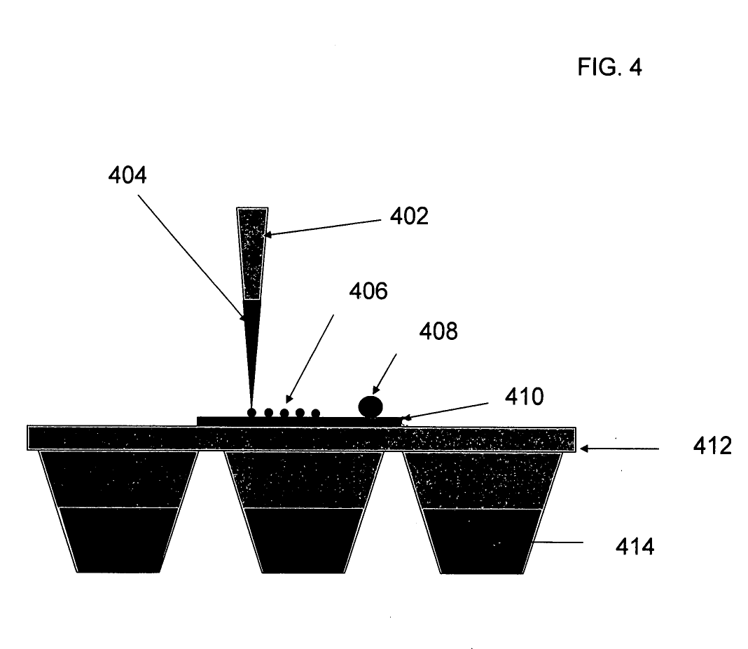

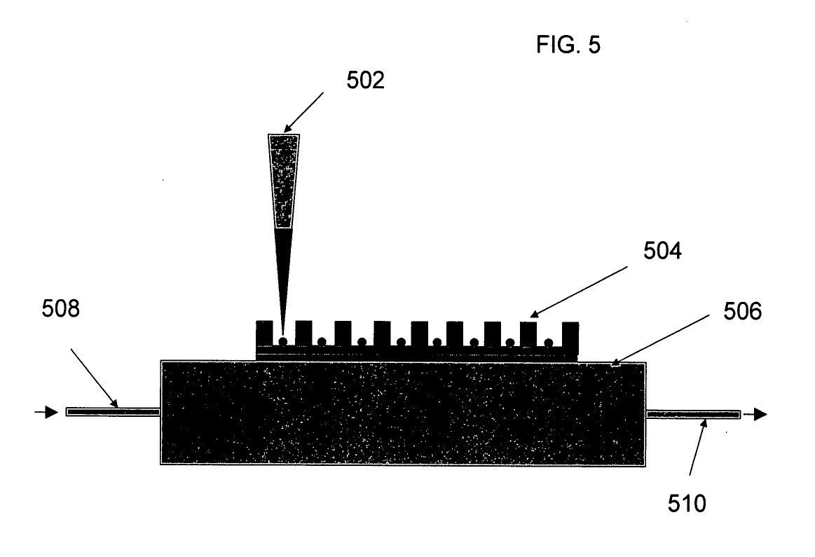

Exemplary apparatus for making lyophilized pellets of the instant invention are further depicted in FIGS. 4 and 5. In FIG. 4 (not shown to size scale), dispense head 402 (e.g., a pipette tip) dispenses fluid 404, such as a biochemical reagent mixture, e.g., PCR master mix, or a sample preparation enzyme mix, a microparticle suspension (e.g., an affinity bead suspension), or a mixture of sample preparation enzyme mix, and microparticle suspension, on to hydrophobic surface 410 (e.g., a diamond-SiO₂ slide deposited by chemical vapour deposition or a Teflon-coated slide). The fluid is dispensed as pellets, shown either as small pellets 406 (e.g., 2 μL volume), or as large pellets (e.g., 25 μL volume). Surface 410 rests upon a support 412 shown in FIG. 4 as, e.g., a “muffin tray” shape, having several declivities in which is a cryogenic agent such as liquid nitrogen 414. An advantage of the muffin tray shape is that it allows the individual wells to be to filled to ¾ full with liquid nitrogen, so that the hydrophobic slide is supported over it without actually being submerged into the liquid. It also thereby allows use of a minimal amount of LN2. It will be apparent that any other grid like structure which is capable of supporting the slide over a LN2 containing vessel will suffice.

FIG. 5 shows another embodiment (not shown to size scale). Dispense head 502 (e.g., controlled by a robot), dispenses fluid in pellets on a highly hydrophobic dispense/pick-and-place plate 504 for lyophilization, with individual wells for pellets. This plate is suitable for direct placement into a lyophilizer. It can be sealed air tight inside the lyophilizer with a matching lid, not shown, after the lyophilization process is completed. For example, the lid can be brought down into contact with plate 504 by application of a piston inside the lyophilizer. A liquid nitrogen-cooled cryogenic plate 506 has a LN₂ inlet 508 and a vent 510 for nitrogen gas.

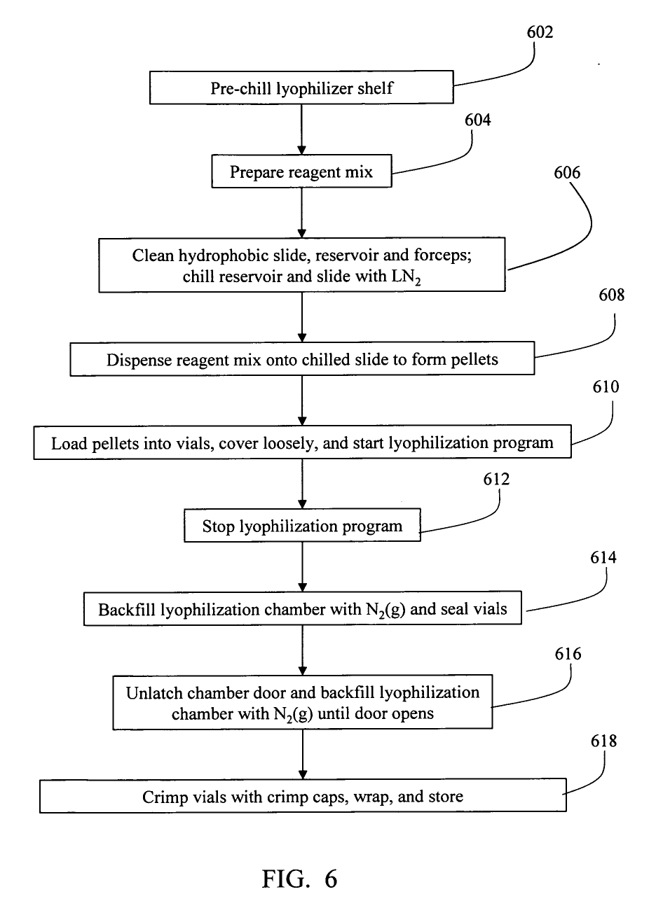

A number of steps in a method of preparing lyophilized pellets according to the present invention are depicted in FIG. 6. At step 602, for efficiency the shelf in the lyophilization equipment is pre-chilled while the reagents are being prepared. This can be accomplished with ordinary controls on the lyophilizer. At step 604, the reagent mix is prepared. The reagent mix can be, for example, a 6× PCR mix, or a bulk lysis mix. At step 606, and prior to dispensing the reagent mix, the hydrophobic slide is cleaned, as is the cryogenic reservoir beneath it, and the forceps or other equipment that may be used to handle the pellets. Cleaning of these items may be accomplished by rinsing in a suitable solvent. The items are then chilled by rinsing in liquid nitrogen.

At step 608, the reagent mix is dispensed onto the cleaned, chilled, hydrophobic slide, thereby forming pellets as previously described herein. The pellets are loaded into vials, step 610, which are covered loosely and placed in the lyophilizer. By ‘covered loosely’ is meant that the vials are preferably covered with ‘lyophilization stoppers’ (20 mm butyl rubber 3 prong flange-type vial plugs). The stoppers when half pressed into the vials, have breathing slits on the side to enable lyophilization to proceed.

The lyophilizer preferably has an automatic control that can be pre-programmed with a sequence of conditions to be applied to the pellets. Typical parameters that can be varied include, but are not limited to: temperature, rate of increase or decrease of temperature, pressure, and time for which a particular set of conditions are maintained. One of ordinary skill in the art will appreciate that identical conditions are not likely to be optimal for all pellet materials. Nevertheless, it will be within the capability of one of ordinary skill in the art to adjust the control cycle for the lyophilizer so that the best quality pellets are obtained.

Typical lyophilizers used in the art, and suitable for use with the present invention include, but are not limited to: Virtis Advantage XL Benchtop Freeze Dryer, and the Virtis Genesis 25 Super XL Pilot Scale Freeze Dryer (both by Virtis, of Gardiner, N.Y.).

Once complete, the lyophilization program stops 412, and the chamber is back-filled with nitrogen gas, to a pressure of, say, 500 Torr. The vials are sealed while still inside the lyophilizer. This can be accomplished because the vials are typically loosely capped, e.g., with lyophilization stoppers which allow the material to easily breathe, and because the lyophilizer itself contains a plate that can be controlled hydraulically, or is powered by compressed dry nitrogen. The plate can be lowered within the lyophilizer to completely stopper the vials prior to opening the door. The lyophilization chamber door is then unlatched, and the pressure inside the chamber increased by back-filling with further nitrogen gas, until the door is forced open. The sealed vials are then finished, e.g., crimped with crimp caps, wrapped in a covering such as aluminum foil, and stored for future use. It is preferable to store the vials at a low temperature, e.g., 4° C. to prolong the lifetime of the pellets.

In general, the concentrations of the compounds in the solution from which the pellets are made is higher than when reconstituted. This is particularly true when the pellets are reconstituted in a microfluidic device. Typically, the ratio of the solution concentration to the reconstituted concentration is at least about 3 (e.g., at least about 4.5). In some embodiments of PCR pellets, the ratio is about 6. Preferably for sample preparation pellets, the ratio is between about 2 and about 20.

Advantageously the lyophilized pellets of the present invention are deployed within a microfluidic cartridge, such as is described in international application serial no. PCT/US2005/015345, filed May 3, 2005, which is incorporated herein by reference in its entirety. For example, certain lyophilized pellets for use in microfluidic devices contain PCR reagents and do not have any microparticles therein. Other lyophilized pellets contain microparticles that are coated with agents that can preferentially capture nucleic acid molecules. Still other lyophilized pellets contain one or more enzymes for different applications but no microparticles. Since the microparticles are used in connection with many applications, but the enzymes change for different applications, it can be convenient in certain circumstances to use lyophilized pellets that contain both the microparticles and the enzymes.

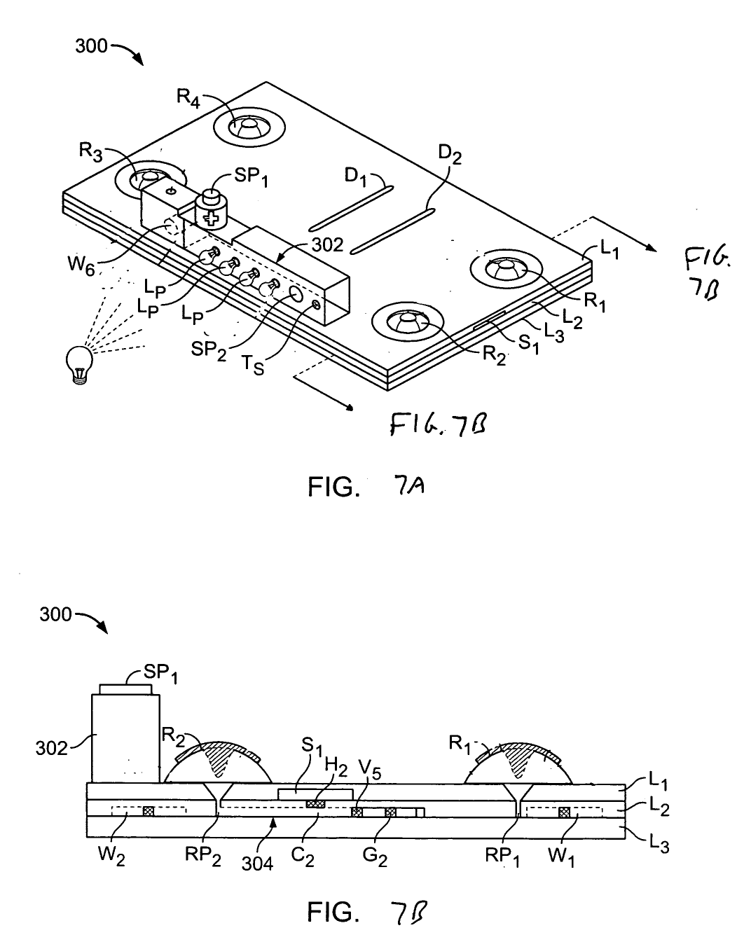

An exemplary microfluidic cartridge is depicted in FIGS. 7A and 7B. Although microfluidic cartridge 300, as shown, is configured to receive polynucleotides already released from cells, other microfluidic devices can be configured to release polynucleotides from cells (e.g., by lysing the cells). For example, microfluidic device 300 in FIGS. 7A and 7B includes a sample lysing chamber 302 in which cells are lysed to release polynucleotides therein. Lyophilized pellets containing lysing reagents according to the present invention may be present in chamber 302 so that, upon application of heat after introduction of cell-containing sample, the lysing reagents are released and lyse cells in the sample. Microfluidic device 300 further includes substrate layers L₁-L₃, a microfluidic network 304 (only portions of which are shown in FIGS. 7A and 7B), and liquid reagent reservoirs R₁-R₄. Liquid reagent reservoirs R₁-R₄ hold liquid reagents (e.g., for processing sample material) and are connected to network 304 by reagent ports RP₁-RP₄ (RP₃ and RP₄ are not shown).

Network 304 is substantially defined between layers L₂ and L₃ but extends in part between all three layers L₁-L₃. Microfluidic network 304 includes multiple components including channels Cₙ, sample input ports SPₙ, valves Vₙ, gates Gₙ, detection zones Dₙ, processing chambers Dₙ, waste zones Wₙ, vents Hₙ and other components not shown, such as double valves V′ₙ, gas actuators (e.g., pumps) Pₙ, and mixing gates MGₙ. Such components are further described elsewhere, such as in international application serial no. PCT/US2005/015345.

In a microfluidic device, actions of a combination of components such as valves, vents and actuators, causes solutions to contact lyophilized pellets, thereby dissolving the pellets and releasing the reagents into solution. Such dissolution is typically very fast, and occurs in about 2 minutes or less. The portions of solution containing the dissolved reagents may then be further moved around the microfluidic network and caused to contact sample, or to mix with other reagent solutions.

### EXAMPLES

Such abbreviations as used herein are those familiar to one of ordinary skill in the art.

### Example 1

### Reagents for Group B Streptococcus (GBS) Determination

Exemplary lyophilized pellets that include representative reagents for the amplification of polynucleotides associated with group B streptococcus (GBS) bacteria are described herein.

An exemplary solution for preparing lyophilized pellets for use in the amplification of polynucleotides indicative of the presence of GBS can be made by combining a cryoprotectant (e.g., 120 mg of trehalose as dry powder), optionally a bulking agent (such as 12 mg of dextran also as a dry powder), a buffer solution (e.g., 50× GBS PCR buffer, 48 microliters of a solution of 1 M tris-base at pH 8.4, 2.5 M KCl, and 200 mM MgCl₂), a first primer (e.g., 1.92 microliters of 500 micromolar GBS Primer F, available from Invitrogen), a second primer (e.g., 1.92 microliters of 500 micromolar GBS Primer R, available from Invitrogen), a probe (e.g., 1.92 microliters of 250 micromolar GBS Probe—FAM, available from IDT/Biosearch Technologies), a control probe (e.g., 1.92 microliters of 250 micromolar Cal Orange 560, available from Biosearch Technologies), a template plasmid (e.g., 0.6 microliters of a solution of 105 copies plasmid per microliter), a specificity control (e.g., 1.2 microliters of a solution of 10 nanograms per microliter (e.g., about 5,000,000 copies per microliter) Streptococcus pneumoniae DNA (available from ATCC)), PCR reagents (e.g., 4.8 microliters of a 100 millimolar solution of dNTPs, available from Epicenter) and 4.8 microliters of a 20 millimolar solution of dUTPs, available from Epicenter), a bulking agent (e.g., 24 microliters of a 50 milligram per milliliter solution of BSA (Invitrogen)), a polymerase (e.g., 60 microliters of a 5 U per microliter solution of glycerol-free Taq Polymerase, available from Invitrogen/Eppendorf) and a solvent (e.g., water) to make about 400 microliters of solution. About 200 aliquots of about 2 microliters each of this solution are frozen and desolvated according to methods described herein to make 200 pellets. When reconstituted, the 200 pellets make a PCR reagent solution having a total volume of about 2.4 milliliters.

### Example 2

### Lyophilized PCR Reagent Pellets

This example describes the manufacture of 200 lyophilized PCR master mix pellets. FIG. 6 is a flow chart of the general procedure employed in Example 2, which is further exemplified in the following narrative.

The total volume of lyophilization master mix employed was 400 μL. Each pellet had a starting volume of 2 μL and was manufactured at a 6× strength. Thus, each pellet was manufactured to contain reagents for a reaction volume of 12 μL. The total lyophilization mix was calculated for a final working reaction volume of 2.4 mL.

Reagents were assembled while the lyophilizer shelf was pre-chilled to −55° C. for 1 hour. The lyophilizer used was the Virtis Advantage XL Benchtop Freeze Dryer. The door on the lyophilizer was shut to prevent accumulation of frozen condensation on the shelf.

The 6× PCR cocktail was prepared from the reagents in Table 1 as follows, while working in a 4° C. environment and keeping materials on ice. Trehalose powder (120 mg) was carefully weighed into a 1.7 mL clean Eppendorf tube (low DNA binding tubes were used). Frozen 50× PCR buffer was thawed to room temperature, thoroughly vortexed until crystals were no longer present, after which 48 mL of buffer was added to the Eppendorf tube. Subsequently, the remaining components in Table 1 were added serially into the tube. Each component was thoroughly vortexed prior to addition, especially the IC plasmid template and the Streptococcus pneumoniae genomic DNA. After addition of all reagents, distilled deionized H₂O (ddH₂O) was added to make the total volume 400 μL. The mixture was vortexed thoroughly and kept on ice.

A hydrophobic slide (a slide with a layer of laser deposited carbon/silicon dioxide coating material), a “muffin tray” (28 cm×18 cm×38 mm 6-well polytetrafluoroethylene coated tray) and forceps were cleaned by washing serially with ddH₂O, absolute ethanol, isopropanol, absolute ethanol, and finally rinsed once again with ddH₂O. Wet materials were dried with compressed air as needed.

In Table 1, dUTPs is listed as optional because it is used only to prevent carryover contamination where applicable, and is not used in the final product.

One of the central wells of the muffin tray was filled with liquid nitrogen (LN₂). The cleaned hydrophobic slide was placed across the mouth of the LN₂-filled well. Approximately 100 mL of LN₂ was poured over the top of the hydrophobic slide and the slide was allowed to equilibriate with the LN₂ for about 2-3 minutes.

TABLE 1
Reagents and Quantities for 6× PCR buffer

| Reagent | Amount | |
|---|---|---|
| Trehalose | 120 | mg |
| HandyLab GBS PCR Buffer (50×) | 48 | μL |
| Bovine serum albumin (BSA) (50 mg/mL) | 24 | μL |
| Deoxyribonucleotide mix (dNTPs) (100 mM) | 4.8 | μL |
| 2′-Deoxyuridine 5′-Triphosphate (dUTPs) (20 mM) | 4.8 | μL |
| [Optional] | | |
| GBS#1 Primer F (500 μM) | 1.92 | μL |
| GBS#1 Primer R (500 μM) | 1.92 | μL |
| GBS#1 Probe-FAM (250 μM) | 1.92 | μL |
| GBS#1 IC Probe-Cal-560-Orange (250 μM) | 1.92 | μL |
| GBS#1 IC Template-Plasmid (1*10⁶ copies/μL) | 2.4 | μL |
| Streptococcus pneumoniae genomic DNA (1 ng/μL) | 8 | μL |
| Thermostable, glycerol free DNA Polymerase (50 U/μL) | 72 | μL |
| 50 mM MgCl₂ | 3.2 | μL |

A clean, fresh, and sterile 0.2 mL autopipettor tip was placed on an autopipettor. The autopipettor was set and autocalibrated to deliver 2 μL. The PCR 6× mix was drawn into the autopipettor tip without pulling up bubbles. The autopipettor tip was manipulated to avoid touching the insides of the PCR 6× mix Eppendorf tube. The tip was wiped dry with a clean disposable laboratory wipe prior to the dispensing process.

Subsequently, frozen 6× PCR mix pellets were prepared by pipetting 2 μL volumes of the 6× PCR mix onto the hydrophobic slide. The autopippettor tip was held close to the slide without actually touching the slide, and was held so that the tip was as perpendicular to the slide as possible. The 2 μL volumes of PCR mix froze almost instantly into pellets and were almost completely spherical. Periodically, the tip was wiped with a disposable laboratory wipe to ensure that the tip was dry on the outside. When not dispensing, the tip was kept sufficiently far from the LN₂ to avoid freezing the PCR mix in the tip. Incorrectly dispensed or malformed pellets were recovered with a pair of forceps, put back into the master mix, and vortexed thoroughly prior to re-dispensing. The tip was again wiped dry with a clean disposable laboratory wipe prior to re-dispensing. In this manner, all of the PCR mix was formed into frozen pellets. Periodically, the muffin tray well was refilled with LN₂ to keep the pellets frozen. However, at no time did either the reagent mix or the pellets come into contact with the LN₂ during formation.

Lyophilization vials were prepared by labeling 40 20 mL borosilicate glass vials with the title “6× PCR Pellet”, lot number, and date. The labeled lyophilization vials were placed into the adjacent wells of the muffin tray and the wells were filled with LN₂. Subsequently, 5 pellets were sequentially loaded into each LN₂ filled lyophilization vial, using a pair of forceps. The forceps' tips were chilled by frequent dipping into the LN₂. Fully loaded vials were kept in a styrofoam box containing 2-5 cm of LN₂ to ensure that the pellets were always submerged in LN₂ until samples were placed inside the lyophilizer.

The loaded vials were covered loosely with plugs (20 mm butyl rubber 3 prong flange-type vial plugs) so that air could freely be exchanged via the recesses on the sides of the caps. The loaded vials were placed into the lyophilizer and the door of the lyophilizer was immediately closed. The lyophilizer had a glass door which was covered with aluminum foil to protect the PCR pellets from external light.

TABLE 2
Lyophilization program for preparing pellets of 6× PCR Buffer

| Stage (and steps) | | Pressure | Temperature State | Time (min) |
|---|---|---|---|---|
| Pre-chill | | 760 Torr | Shelf at −55° C. | 60 |
| Load Tray | | 760 Torr | Hold at −55° C. | N/A |
| Vacuum | | 760 Torr- | Hold at −55° C. | N/A |
| | | 100 mTorr | | |
| Primary | 1 | 100 mTorr | Hold at −55° C. | 420 |
| Drying | 2 | 100 mTorr | Ramp from −55° C. | 180 |
| | | | to −37° C. | |
| | 3 | 100 mTorr | Hold at −37° C. | 300 |
| | 4 | 100 mTorr | Ramp from −37° C. | 360 |
| | | | to +10° C. | |
| | 5 | 100 mTorr | Hold at +10° C. | 180 |
| | 6 | 100 mTorr | Ramp from +10° C. | 120 |
| | | | to +25° C. | |
| | 7 | 100 mTorr | Hold at +25° C. | 60 |
| Secondary | 8 | 100 mTorr | Hold at +25° C. | 60 |
| Drying | | | | |

The lyophilizer was evacuated to 500 Torr, at which point the automatic lyophilization program shown in Table 2 was initiated beginning at step 1 of “Primary Drying.”

After 28 hours (end of step 8 of “Secondary drying”), the lyophilization process was manually terminated. The chamber was immediately backfilled to a pressure of 500 Torr of dry N₂ by opening the valve on the regulator of an attached low pressure dry N₂ tank (the regulator of which was preset to near 0 kPa). When the chamber reached 500 Torr, the valve for an attached high pressure dry N₂ tank (the regulator of which was preset to approximately 689 kPa) was opened. The lyophilizer was equipped with a stoppering lever, which was operated with sufficient force against the loosely inserted butyl rubber vial plugs to seal the vials with the plugs. The lever was left in the “down” position for 5 seconds to seal the plugs in the vials and then returned to the “up” position. The valve on the high pressure dry N₂ tank was closed. The “vac release” function of the lyophilizer was operated and the lyophilizer chamber door handle was unlatched. Nitrogen was allowed to flow into the chamber from the low pressure tank until the lyophilization chamber door opened. The nitrogen tank valve was closed, the sample vials were removed, and 20 mm red aluminum tear-off seal crimp caps were manually crimped onto the plugs. The vials were stored in a light proof container at 4° C.

### Example 3

### Lyophilized Sample Preparation Pellets

This example describes the manufacture of 1,000 lyophilized sample prep pellets containing enzyme mix (BLP-EM). FIG. 6 is a flow chart of the general procedure employed in Example 3, which is further exemplified in the following narrative.

The total volume of the lyophilization master mix employed was 25 mL. Each pellet had a starting volume of 25 μL. Two pellets are required for each 1 mL lysis reaction. Thus each pellet contained reagents for a reaction volume of 500 μL and the total lyophilization mix was calculated for a final working reaction volume of 1.0 mL.

To process 1 mL of clinical sample, it is preferable to use 4 sample preparation pellets: two each of enzyme pellets and affinity pellets (see Example 4). Therefore, two enzyme pellets in addition to lyophilized sample preparation pellets containing affinity beads constitute the total lyophilization mix for a single 1 mL lysis and DNA binding reaction.

Reagents were assembled while the lyophilizer shelf was pre-chilled to −55° C. for 1 hour. The lyophilizer door was shut to prevent accumulation of frozen condensation on the shelf.

The fresh bulk lysis mix was prepared as follows, while working in a 4° C. environment and keeping materials on ice. Trehalose powder (7.5 g) was carefully weighed into a 50 mL sterile Falcon tube (low DNA binding tubes were used); 18 mL of ddH₂O was added to the trehalose powder and mixed by vortexing. The components in Table 3 were added sequentially in the amounts shown. Each component was thoroughly vortexed prior to addition.

TABLE 3

| Component | U/1000rxn |
|---|---|
| RNase A | 18,000 |
| Pronase | 5600 |
| Proteinase K | 12,000 |
| Mutanolysin | 75,000 |

After addition of all reagents, ddH₂O was added to make the total volume 25 mL. The bulk lysis mix was vortexed thoroughly and kept on ice.

Hydrophobic slides (slides with a layer of laser deposited carbon/silicone dioxide coating material), a muffin tray (28 cm×18 cm×38 mm 6-well polytetrafluoroethylene coated tray) and forceps were cleaned by washing serially with ddH₂O, absolute ethanol, isopropanol, absolute ethanol, and finally rinsed once again with ddH₂O. Wet materials were dried with compressed air as needed.

All of the wells of the muffin tray were filled to the brim with LN₂. A cleaned hydrophobic slide was placed across the mouth of each LN₂-filled well. Approximately 100 mL of LN₂ was poured over the top of each hydrophobic slide and the slides were allowed to equilibrate with the LN₂ for about 2-3 minutes.

A clean, fresh, and sterile 0.5 mL autopipettor tip was placed on an autopipettor. The autopipettor was set and autocalibrated to deliver 25 μL. The bulk lysis mix was drawn into the autopipettor tip without pulling up bubbles. The autopipettor tip was manipulated to avoid touching the insides of the bulk lysis mix source container. The tip was wiped dry with a clean disposable laboratory wipe prior to the dispensing process.

Subsequently, frozen bulk lysis mix pellets were prepared by pipetting 25 μL volumes of the bulk lysis mix onto a hydrophobic slide. The autopipettor tip was held close to the slide without actually touching the slide, and was held so that the tip was as perpendicular to the slide as possible. The 25 μL volumes of bulk lysis mix froze almost instantly at the bottom of the drop and froze slowly towards the top until the entire drop was frozen. Periodically, the pipette tip was wiped with a disposable laboratory wipe to ensure that the tip was dry on the outside. When not dispensing, the tip was kept sufficiently far from the LN₂ to avoid freezing the bulk lysis mix in the tip. Incorrectly dispensed or malformed pellets were recovered with a pair of forceps, put back into the master mix, and vortexed thoroughly prior to re-dispensing. The tip was again wiped dry with a clean disposable laboratory wipe prior to re-dispensing. In this manner, all of the bulk lysis mix was formed into frozen pellets. Periodically, the muffin tray wells were refilled with LN₂ to keep the slide cold and pellets frozen.

Lyophilization vials were prepared by labeling 50 20 mL borosilicate glass vials with the title “BLP-EM”, lot number, and date. The labeled lyophilization vials were placed into the adjacent wells of the muffin tray and filled with LN₂. Subsequently, 20 pellets were sequentially loaded into each LN₂ filled lyophilization vial, using a pair of forceps. The forceps' tips were chilled by frequent dipping into the LN₂. Fully loaded vials were kept in a styrofoam box containing 2-5 cm of LN₂ to ensure that the pellets were always submerged in LN₂ until samples were placed inside the lyophilizer.

The loaded vials were covered loosely with plugs (20 mm butyl rubber 3 prong flange-type vial plugs) so that air could freely be exchanged via the recesses on the sides of the caps. The loaded vials were placed into the lyophilizer and the door of the lyophilizer was immediately closed. The lyophilizer had a glass door which was covered with aluminum foil to protect the bulk lysis mix from external light.

The lyophilizer was evacuated to 500 Torr, at which point the automatic lyophilization program shown in Table 4 was initiated beginning at step 1 of “Primary Drying.”

TABLE 4
Lyophilization program for Example 3

| Stage (and steps) | | Pressure | Temperature State | Time (min) |
|---|---|---|---|---|
| Pre-chill | | 760 Torr | Shelf at −55° C. | 60 |
| Load Tray | | 760 Torr | Hold at −55° C. | N/A |
| Vacuum | | 760 Torr- | Hold at −55° C. | N/A |
| | | 100 mTorr | | |
| Primary Drying | 1 | 100 mTorr | Hold at −55° C. | 420 |
| | 2 | 100 mTorr | Ramp from −55° C. | 120 |
| | | | to −37° C. | |
| | 3 | 100 mTorr | Hold at −37° C. | 480 |
| | 4 | 100 mTorr | Ramp from −37° C. | 360 |
| | | | to +10° C. | |
| | 5 | 100 mTorr | Hold at +10° C. | 240 |
| | 6 | 100 mTorr | Ramp from +10° C. | 120 |
| | | | to +25° C. | |
| | 7 | 100 mTorr | Hold at +25° C. | 30 |
| Secondary | 8 | 100 mTorr | Hold at +25° C. | 30 |
| Drying | | | | |

After 30 hours (end of step 8 “Secondary drying” in Table 4), the lyophilization process was manually terminated. The chamber was immediately backfilled to a pressure of 500 Torr of dry N₂ by opening the valve on the regulator of an attached low pressure dry N₂ tank (the regulator of which was preset to near 0 kPa). When the chamber reached 500 Torr, the valve for an attached high pressure dry N₂ tank (the regulator of which was preset to approximately 689 kPa) was opened. The lyophilizer was equipped with a stoppering lever, which was operated with sufficient force against the loosely inserted butyl rubber vial plugs to seal the vials with the plugs. The lever was left in the “down” position for 5 seconds to seal the plugs in the vials and then returned to the “up” position. The valve on the high pressure dry N₂ tank was closed. The “vac release” function of the lyophilizer was operated and the lyophilizer chamber door handle was unlatched. Nitrogen was allowed to flow into the chamber from the low pressure tank until the lyophilization chamber door opened. The nitrogen tank valve was closed, the sample vials were removed, and 20 mm red aluminum tear-off seal crimp caps were manually crimped onto the plugs. The vials were stored in a light proof container at 4° C.

### Example 4

### Lyophilized Microparticle-Containing Pellets

Lyophilized pellets containing DNA affinity microspheres were made up using substantially the same procedures as outlined in Example 4, and FIG. 6, with the exception that the compositions employed are shown in Table 5, and the method of preparing a microparticle suspension is as follows.

TABLE 5
Reagents and Amounts for Microparticle pellets

| Component | Quantity | |
|---|---|---|
| Microspheres | 15.0 | mL |
| Trehalose | 7.5 | g |

7.5 g of Trehalose powder was carefully weighed out into a 50 mL sterile Falcon Tube. Affinity Beads were prepared as follows: 15.0 mL micro-spheres were pipetted into a 50 mL sterile Falcon tube. The micro-spheres were centrifuged to pellet in a swinging bucket rotor at 3,500 rpm for 15 minutes. The supernatant was carefully and completely removed and discarded. The micro-spheres were resuspended in 10.0 mL ultrapure water and vortexed thoroughly. The resuspended PSPDL micro-spheres were added to the trehalose plus an additional 8.0 mL of ultrapure water. The mixture was vortexed until all trehalose has completely dissolved.

The volume of the mix was made up to 25 mL with ultrapure water. (The water can be brought up to 25 mL by taking up all the mix into a 25 mL pipette and dispensing until the liquid reaches the tip. The difference in volumes should be added to the mix).

The preparation of lyophilized pellets now proceeds by vortexing the mix thoroughly and keeping the same on ice, before proceeding at step 606 of FIG. 6.

### Example 5

### Reagents for Determination of Pathogens

PCR pellets have been manufactured for detection of Yersinia pestis (plague), Erwinia herbicola (a plant pathogen often used as a plague stimulant), Bacillus anthracis, and Bacillus globigii (an anthrax stimulant), according to the formulation of Example 1, except that the primers for the respective pathogen are substituted for the GBS primer(s) and probe. PCR pellets can also be manufactured for Listeria monocytogenes, E. coli O157, and Herpes Simplex Virus 1 & 2, according to analogous formulations.

A number of embodiments of the invention have been described. Nevertheless, it will be understood that various modifications may be made without departing from the spirit and scope of the invention. For example, additional or alternative reagents may be employed within the lyophilized pellets of the present invention. Accordingly, other embodiments are within the scope of the following claims.

What is claimed is:

1. A lyophilized pellet, comprising:
a cryoprotectant;
a PCR reagent mix;
a salt selected from the group consisting of: KCl, MgCl₂, and (NH₄)₂SO₄; and
optionally a buffering agent;
wherein the lyophilized pellet has a volume in the range 0.5 to 35 μL.

2. A lyophilized pellet according to claim 1, wherein the PCT reagent mix comprises:
at least one enzyme;
at least one protein;
at least one primer;
at least one fluorogenic probe;
at least one plasmid;
at least one polypeptide; and
at least one nucleotide selected from the group consisting of: dATP, dGTP, dCTP, dTTP, and dUTP; and
optionally at least one non-specific control nucleic acid.

3. A lyophilized pellet according to claim 1, having a buffering agent, wherein the buffering agent is Tris.

4. A lyophilized pellet according to claim 1, wherein the cryoprotectant is trehalose.

5. A lyophilized pellet according to claim 1, additionally comprising a bulking agent.

6. A method for making a lyophilized pellet, comprising:
introducing a liquid composition into a dispensing tip;
positioning the dispensing tip above a cryogenically cooled plate, wherein the plate has a hydrophobic surface, and wherein the tip is in close proximity to the surface;
dispensing a droplet of the liquid composition from the tip, on to the surface, wherein the droplet is momentarily in contact with both the tip and the surface;
removing the tip away from close proximity to the surface so that the droplet remains in contact with the surface;
maintaining the droplet in contact with the surface for such time as the droplet freezes to form a frozen droplet; and
placing the frozen droplet into a lyophilizer under conditions sufficient to produce a lyophilized pellet.

7. The method of claim 6 wherein the cryogenically cooled plate has a temperature of about −65° C. to −180° C.

8. The method of claim 6 wherein the hydrophobic surface is essentially flat.

9. The method of claim 6 wherein the hydrophobic surface is selected from the group consisting of: a diamond surface; a silicon oxide surface; a diamond-SiO₂ slide; and PTFE.

10. The method of claim 6 wherein said method is multiplexed, comprising a number of dispensing tips, thereby producing a number of lyophilized pellets simultaneously.

11. The method of claim 6 wherein the lyophilized pellet is essentially spherical.

12. The method of claim 6 wherein the liquid composition comprises a concentration of microspheres.

13. The method of claim 12 wherein the concentration is in the range of 10³ to 10¹³ microspheres per mL.

14. The method of claim 12 wherein the concentration is 10⁶-10⁸ microspheres per mL.

15. A lyophilized pellet according to claim 12, containing between about 1 and about 10¹² microspheres.

16. The method of claim 6 wherein the microspheres are coated with a polycationic material.

17. The method of claim 6 wherein the liquid composition comprises a mixture of biological reagents selected from the group consisting of: a mixture of enzymes; and a PCR reagent mix.

18. The method of claim 6 wherein close proximity to the surface is between 0.5 and 1.5 mm.

19. The method of claim 6 wherein the lyophilized pellet has a volume between 0.5 and 35 μL.

20. The method of claim 6 wherein the lyophilized pellet has a volume between 2 and 25 μL.

21. The method of claim 6 wherein the lyophilized pellet has a volume between 1 and 10 μL.

22. The method of claim 6, wherein the conditions sufficient to produce a lyophilized pellet include a period of residency in the lyophilizer from about 20 to 40 hours.

23. The method of claim 6 wherein the lyophilized pellet has a diameter between about 0.5 and about 5 mm.

24. A lyophilized pellet made by the method of claim 6 having a sphericity between 0.75 and 1.

25. An apparatus for preparing lyophilized pellets, comprising:
a cryogenically cooled plate having a hydrophobic surface;
a dispensing system configured to position a dispensing tip above and in proximity to the hydrophobic surface, wherein the dispensing tip is configured to dispense a droplet of liquid onto the hydrophobic surface causing a frozen droplet to form; and
a chamber enclosing at least the hydrophobic surface, configured to apply conditions of temperature and pressure sufficient to lyophilize the frozen droplet.

26. A microfluidic cartridge, comprising:
a reagent inlet, wherein are situated one or more lyophilized pellets that each contain one or more reagents; and
a microfluidic network, having at least one microfluidic channel in communication with the reagent inlet, wherein the channel is configured to permit fluid to pass from the inlet into the channel;
wherein the one or more lyophilized pellets have a composition according to claim 1.

27. The cartridge of claim 26, further comprising:
a lysis chamber, wherein are situated one or more lyophilized pellets that each contain one or more lysis reagents.

28. The cartridge of claim 26, wherein the network further comprises at least one component selected from the group consisting of: a valve; a gate; an outlet; a vent; and a channel.

29. A lyophilized pellet, comprising:
a cryoprotectant; and
a plurality of microspheres having a concentration in the range of 10³ to 10¹³ per mL;
wherein the lyophilized pellet has a volume in the range 0.5 to 35 μL.

30. A lyophilized pellet according to claim 29, having an essentially spherical shape.

31. A lyophilized pellet according to claim 29 wherein the microspheres are coated with a polycationic material.

32. A lyophilized pellet according to claim 31, wherein the polycationic material is selected from the group consisting of: poly-D-lysine; polyethyleneimine; poly-DL-ornithine; and poly-histidine.

33. A lyophilized pellet according to claim 32 wherein the polycationic material is poly-D-lysine whose constituent molecules have molecular weights between 1,000 and 4,000 Daltons.

34. A lyophilized pellet according to claim 33, wherein the poly-D-lysine has an average molecular weight of about 1770 Daltons.

35. A lyophilized pellet according to claim 32 wherein the polycationic material is polyethyleneimine whose constituent molecules have molecular weights between 600 and 800 Daltons.

36. A lyophilized pellet according to claim 32 wherein the polycationic material is poly-DL-ornithine whose constituent molecules have molecular weights between 12,000 and 30,000 Daltons.

37. A lyophilized pellet according to claim 29, additionally comprising a bulking agent.

38. A lyophilized pellet according to claim 29, wherein the cryoprotectant is trehalose.

39. A lyophilized pellet according to claim 29, additionally comprising a mixture of enzymes.

40. A lyophilized pellet, comprising:
a cryoprotectant;
a primer;
a probe;
an internal control plasmid;
a specificity control;
a PCR reagent;
optionally a salt;
optionally a bulking agent; and
a polymerase.

41. A lyophilized pellet according to claim 39, wherein:
the primer, the probe, and the specificity control, are specific to Group B Streptococcus.

42. A lyophilized pellet according to claim 39, wherein:
the primer, the probe, and the specificity control, are specific to a pathogen selected from the group consisting of: Yersinia pestis; Erwinia herbicola; Bacillus anthracis; Bacillus globigii; Listeria monocytogenes; E. coli O157; Herpes Simplex Virus 1; and Herpes Simplex Virus 2.
